(19) United States
(12) Patent Application Publication
(10) Pub. No.: US 2019/0330150 A1
(43) Pub. Date: Oct. 31, 2019

(54) Processes for the Preparation of 3-(4-Halobutyl)-5-Cyanoindole

(71) Applicant: Apotex Inc., Toronto (CA)

(72) Inventors: Gamini Weeratunga, Ancaster (CA); Avedis Karadeolian, Cambridge (CA); Prabhudas Bodhuri, Torrance, CA (US); Dineshkumar N. Patel, Brantford (CA); Boris Gorin, Oakville (CA)

(21) Appl. No.: 16/391,541

(22) Filed: Apr. 23, 2019

Related U.S. Application Data
(60) Provisional application No. 62/664,501, filed on Apr. 30, 2018.

(51) Int. Cl. C07D 209/10; B01J 19/18

(52) U.S. Cl. CPC C07D 209/10; B01J 2219/00051; B01J 2219/00761; B01J 19/1862

(57) ABSTRACT

The present invention provides continuous flow processes for the preparation of the compound of Formula (1), an intermediate in the preparation of Vilazodone.

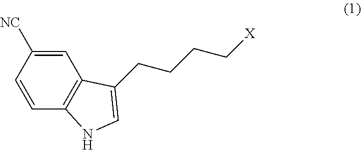

### CROSS-REFERENCE TO RELATED APPLICATION

This application claims the benefit of U.S. Provisional Patent Application No. 62/664,501, filed Apr. 30, 2018, the disclosure of which is hereby incorporated by reference in its entirety.

### TECHNICAL FIELD

The present invention relates to processes for the preparation of intermediates useful in the preparation of Vilazodone, and in particular, a 3-(4-halobutyl)-5-cyanoindole.

### BACKGROUND

Vilazodone hydrochloride, or 5-[4-[4-(5-cyano-1H-indol-3-yl)butyl]-1-piperazinyl]-2-benzofurancarboxamide hydrochloride, exhibits activity as a selective serotonin reuptake inhibitor (SSRI), and is marketed in the United States as VIIBRYD®, which is indicated for the treatment of patients with major depressive disorder (MDD). Vilazodone hydrochloride (A) has the following structural formula:

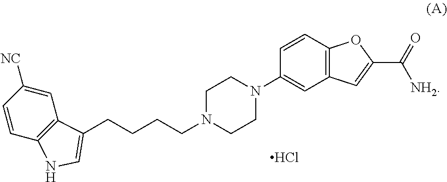 [SMILES: N#Cc1ccc2[nH]cc(CCCCN3CCN(c4ccc5oc(C(N)=O)cc5c4)CC3)c2c1 ; Cl]

The compound of Formula (1), wherein X is a halide, and particularly wherein X is chloride, is reported to be useful as an intermediate in the preparation of Vilazodone hydrochloride (A):

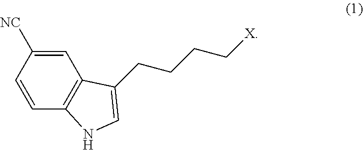

One approach that is commonly used to prepare the compound of Formula (1) is by reductive deoxygenation of the compound of Formula (2) in the presence of a suitable reducing agent as shown in Scheme 1. This methodology is reported in, for example, U.S. Pat. Nos. 5,418,237, 5,532,241, 6,509,475, Heinrich, T., et al. J. Med. Chem. 2004, 47, 4684-4692, Heinrich, T., et al. ACS Med. Chem. Lett. 2010, 1, 199-203, CN102690224, CN102659660, WO 2014/006637 A2, CN102875440, CN103467357, WO 2013/153492 A2, CN103880729, CN104592087, CN103910668 and U.S. Pat. No. 9,533,949.

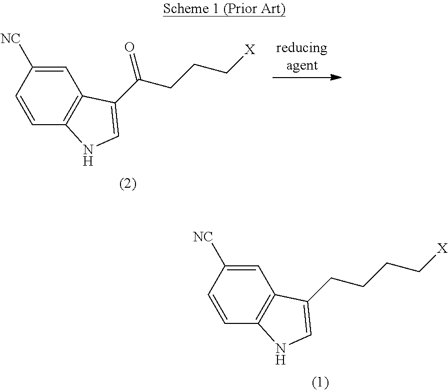

In many cases, known processes for this reduction employ hazardous reducing agents and Lewis acids such as diborane, sodium bis(2-methoxyethoxy)aluminium hydride (Red-Al®), chlorotrimethylsilane, isobutylaluminium dichloride and titanium tetrachloride. Further, the use of highly reactive and pyrophoric reagents like diborane and isobutylaluminium dichloride raises safety concerns, particularly when used on an industrial scale, where the control of exotherms of a large volume of material is challenging. When using such reagents, the necessary thermal control is typically achieved by slow addition of the reactive agent(s) under the application of a cooling mechanism, which is inefficient on a large scale owing to the low surface area to volume ratio of the reducing agent and the compound of Formula (2), and a reduction in mixing effectiveness. Additionally, depending on the choice of reagents, over-reduction of the indole ring has been reported to occur using this approach.

Owing to the drawbacks of the existing processes for the preparation of the compound of Formula (1), there is a need for improved processes for the preparation of the compound of Formula (1) that are more amenable to scale-up and use in a commercial setting.

### SUMMARY OF THE INVENTION

The present invention provides improved processes for the preparation of the compound of Formula (1) that employ continuous flow technology.

Development of the processes of the present invention followed, in part, from the industrial scaling of the batch-mode reduction using sodium borohydride/iron(III) chloride that is described in U.S. Pat. No. 9,533,949 B2 and shown in Scheme 2. When this reduction was scaled from laboratory to industrial scale, there was an increase in the amount of certain impurities, and in particular, the impurity of Formula (IMP), which occurred at levels of 2-3%, and was persistent and difficult to remove. As a result, additional purification operations were required to lower the impurity levels in the compound of Formula (1-A) to acceptable levels, resulting in a lower than desired overall yield. Methods were therefore desired to provide the compound of Formula (1), and particularly the compound of Formula (1-A), with an improved impurity profile and increased yield, goals that were achieved with the continuous flow process described herein.

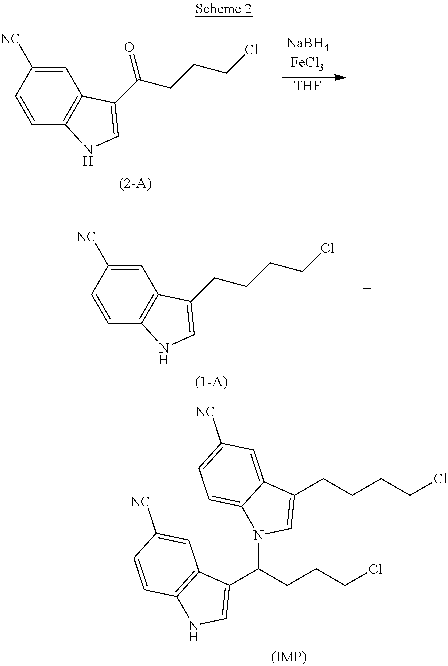 [SMILES: N#Cc1ccc2[nH]cc(C(=O)CCCCl)c2c1 ; N#Cc1ccc2[nH]cc(CCCCCl)c2c1 ; N#Cc1ccc2[nH]cc(C(CCCCl)n3cc(CCCCCl)c4cc(C#N)ccc43)c2c1 ; "THF"]

Without wishing to be bound to any particular theory, it is believed that the impurity of Formula (IMP) forms as a result of the reaction shown in Scheme 3, wherein the intermediate of Formula (3-A) reacts with the product of Formula (1-A). Based on this potential mechanism, it was reasoned that lower levels of the impurity of Formula (IMP) may be attainable by limiting the exposure of the intermediate of Formula (3-A) to the product of Formula (1-A). Thus, the present invention provides a process for the preparation of the compound of Formula (1-A) that produces lower levels of impurity of Formula (IMP) than are observed in known processes, by limiting the exposure of the intermediate of Formula (3-A) to the product of Formula (1-A) through the use of a continuous flow process such that reaction materials are carried as a flowing stream, with reactants/reagents continuously streamed in and products continuously streamed out. In principle, when compared to a batch process, the continuous flow processes of the present invention provide greater separation between different phases of the reaction, such as starting materials, intermediates and products, thereby lowering the frequency of side-reactions and impurity formation. Indeed, preferred embodiments of the present invention provide the compound of Formula (1-A) having less than 0.1% of the impurity of Formula (IMP) prior to any purification operations in yields as high as 71 Scheme 3

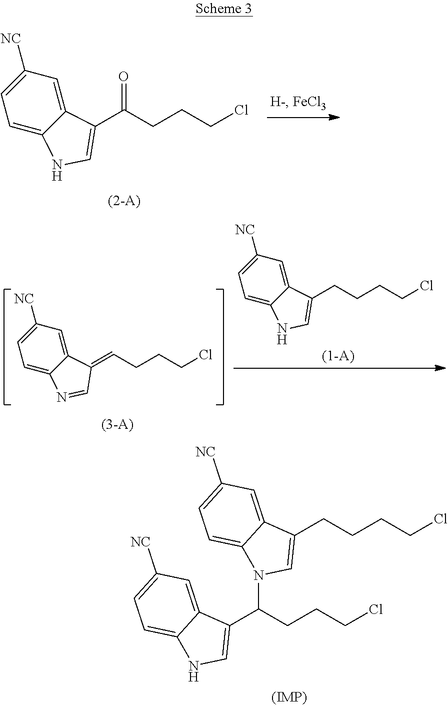

In addition to the increased reaction selectivity provided by the processes of the present invention, the processes also provide increased safety arising from the reduced accumulation of potentially unstable and reactive intermediates associated with the use of reactive reducing agents, and to the increased ability to control exotherms that is provided by the high surface area to volume ratio in the flow system. In addition, effective heat transfer and mixing allows for enhanced temperature control, wherein cooling can be applied very briefly to control exotherms for safety reasons, for example, followed immediately by a period of heating to the optimal reaction temperature to facilitate the desired reaction.

Accordingly, in a first aspect of the present invention, there is provided a continuous flow process for the preparation of the compound of Formula (1):

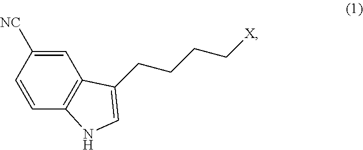

comprising contacting a continuous flow (F1) of the compound of Formula (2):

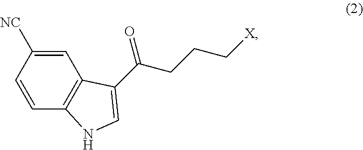

wherein X is a halide,
in a solvent (S1), with a continuous flow (F2) of a Lewis acid in a solvent (S2), and with a continuous flow (F3) of a hydride donor reducing agent in a solvent (S3), to provide continuous flow (F4) containing the compound of Formula (1).

In a preferred embodiment of the first aspect, continuous flow (F1) and continuous flow (F2) are combined to form combined continuous flow (F1-2) prior to contact with continuous flow (F3).

In a further preferred embodiment of the first aspect, continuous flows (F1) and (F2) are combined in a first reactor, and the combined continuous flow (F1-2) that exits the reactor is contacted with continuous flow (F3) in a second reactor downstream from the first reactor to provide the continuous flow (F4). More preferably, continuous flow (F4) passes from the second reactor to a third reactor downstream from the second reactor. Preferably, the reactors are continuous stirred tank reactors.

In a further preferred embodiment of the first aspect, continuous flows (F1) and (F2) are combined at a first intersection to provide combined continuous flow (F1-2) that is then contacted with continuous flow (F3) at a second intersection downstream from the first intersection to provide continuous flow (F4). More preferably, continuous flow (F4) passes through one or more reactors connected in series downstream from the second intersection. Preferably, the reactors are continuous stirred tank reactors.

In another preferred embodiment of the first aspect, continuous flow (F4) is contacted with a supplemental continuous flow (F3′) of the hydride reducing agent in the solvent (S3′).

In preferred embodiments of the first aspect, X is chloride.

In further preferred embodiments of the first aspect, the hydride donor reducing agent is selected from the group consisting of lithium aluminium hydride, potassium borohydride, sodium borohydride, sodium cyanoborohydride, triethylsilane, borane, diisobutylaluminium hydride and sodium bis(2-methoxyethoxy)aluminium hydride.

In further preferred embodiments of the first aspect, the Lewis acid is selected from the group consisting of aluminium(III) chloride, iron(III) chloride, magnesium chloride, zinc chloride and boron trifluoride.

In preferred embodiments of the first aspect, the hydride donor reducing agent is selected from the group consisting of borane and sodium borohydride and the Lewis acid is iron(III) chloride.

In further preferred embodiments of the first aspect, each of the solvents (S1), (S2), (S3) and, if present, (S3′), is independently an ether solvent.

In a further preferred embodiment of the first aspect, the Lewis acid is iron(III) chloride, the hydride donor reducing agent is sodium borohydride, solvents (S1) and (S2) are both tetrahydrofuran, and solvent (S3) is tetraglyme. In another preferred embodiment, the Lewis acid is iron(III) chloride, the hydride donor reducing agent is borane, and each of the solvents (S1), (S2). (S3) and, if present, (S3′), is tetrahydrofuran.

In another preferred embodiment of the first aspect, continuous flow (F4) is quenched prior to isolation of the compound of Formula (1). Preferably, continuous flow (F4) is quenched using an aqueous solution. Most preferably, the aqueous solution comprises citric acid.

In a second aspect of the present invention, there is provided a continuous flow system for conducting the continuous flow process of the first aspect of the invention. In a preferred embodiment, the continuous flow system comprises:
a first vessel for holding the solution of the compound of Formula (2) in solvent (S1), wherein the first vessel is in fluid communication with one end of a first conduit;
a second vessel for holding the solution of the Lewis acid in solvent (S2), wherein the second vessel is in fluid connection with one end of a second conduit; and
a third vessel for holding the solution of the hydride donor reducing agent in solvent (S3), wherein the third vessel is in fluid communication with one end of a third conduit;
   wherein
the first, second and third conduits are merged at their second ends via one or more intersections to provide a fourth conduit that is in fluid communication with a quench tank; and
one or more pumps cause the continuous flow of a first continuous flow of solution from the first vessel, a second continuous flow of solution from the second vessel, and a third continuous flow of solution from the third vessel through the conduits of the continuous flow system to the quench tank.

In a preferred embodiment of the second aspect, the first and second conduits are merged at a first intersection to provide a fifth conduit, and the third and fifth conduits are merged at a second intersection to provide the fourth conduit, and each of the intersections comprises either one or more reactors allowing for the mixing of two continuous flows, or three-way joints allowing for the merger of two continuous flows into one continuous flow. In a further preferred embodiment, each of the first and second intersections comprises a reactor. Alternatively, each of the first and second intersections comprises a three-way joint.

In a further preferred embodiment of the second aspect, the fourth conduit comprises one or more reactors connected in series between the second intersection and the quench tank.

In another preferred embodiment of the second aspect, the continuous flow system further comprises a supplemental vessel for holding a supplemental portion of the hydride donor reducing agent in solvent (S3′), wherein the supplemental vessel is in fluid communication with a supplemental conduit at one end, and another end of the supplemental conduit merges with the fourth conduit at a third intersection comprising either a reactor or a three-way joint, and a pump causes the continuous flow of solution from the supplemental vessel through the supplemental conduit.

### BRIEF DESCRIPTION OF THE DRAWINGS

Embodiments of the present invention are described, by way of example only, with reference to the attached Figures.

FIG. 1 provides a schematic of a continuous flow system used in the processes of the present invention, as Exemplified in Examples 1 and 2.

FIG. 2 provides a schematic of an alternative continuous flow system used in the processes of the present invention, as exemplified in Example 3.

FIG. 3 provides a schematic of an alternative continuous flow system of the present invention comprising a plug flow reactor (“PFR”).

### DETAILED DESCRIPTION

The processes of the present invention provide improvements in the preparation of the compound of Formula (1), which is an intermediate useful in the preparation of Vilazodone (A), over known processes, including enhanced safety, efficiency and selectivity, and are therefore more amenable to industrial application.

As used herein, “room temperature” generally refers to a temperature of 20-25° C.

As used herein, the term “about” means “close to”, and that variation from the exact value that follows the term is within amounts that a person of skill in the art would understand to be reasonable. For example, when the term “about” is used with respect to temperature, a variation of ±5° C. is generally acceptable when carrying out the processes of the present invention. When used with respect to mole equivalents, a variation of ±0.1 moles is generally acceptable.

As used herein, the term “conduit” refers to any compatible pipe, tube, channel or similar means for conveying fluids in a continuous flow process.

As used herein, the term “residence time” refers to the time required for the subject material to traverse a specified pathway.

As used herein, the term “continuous stirred tank reactor”, abbreviated “CSTR” refers to a reactor comprising a tank, a stirring system and means to continuously introduce reactants and continuously remove products.

As used herein, the term “plug flow reactor”, abbreviated “PFR”, refers to a reactor comprising a conduit wherein reactants are continuously introduced through an inlet and products are continuously withdrawn through an outlet.

In one embodiment of the present invention, there is provided a continuous flow process for the preparation of the compound of Formula (1):

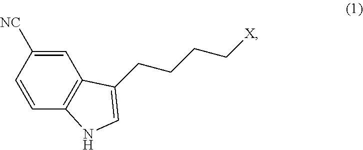

comprising contacting a continuous flow (F1) of the compound of Formula (2):

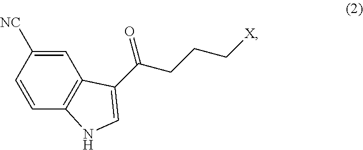

wherein X is halide,
in a solvent (S1), with a continuous flow (F2) of a Lewis acid in a solvent (S2), and with a continuous flow (F3) of a hydride donor reducing agent in a solvent (S3), to provide continuous flow (F4) containing the compound of Formula (1).

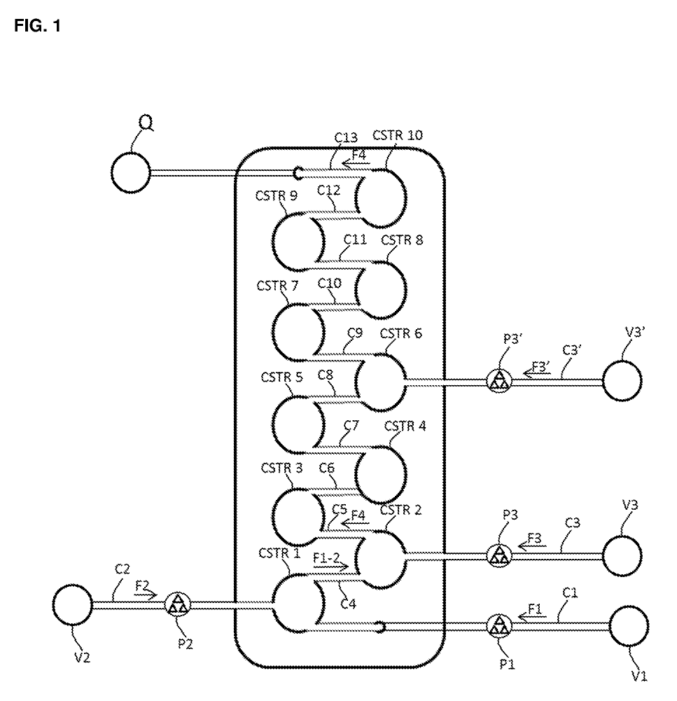

In preferred embodiments of the present invention, the continuous flow process is executed in continuous flow systems having configurations such as those shown in FIGS. 1 to 3. In these configurations, stock preparations of the compound of Formula (2), the Lewis acid, and the hydride donor reducing agent in the respective solvents (S1), (S2) and (S3) are provided in respective vessels (V1), (V2) and (V3), having associated conduits (C1), (C2) and (C3) for the passage of each of the respective stock preparations as continuous flows (F1), (F2) and (F3). Optionally, a supplementary stock preparation of the hydride donor reducing agent in solvent (S3′) is provided in vessel (V3′) having associated conduit (C3′) for passage of the preparation as continuous flow (F3′). The continuous flows (F1), (F2), (F3) and, when utilized, (F3′), combine to provide continuous flow (F4) containing the compound of Formula (1). In preferred embodiments of the invention, continuous flow (F4) is collected into a quench tank (Q) prior to the isolation of the compound of Formula (1).

In FIG. 1, vessels (V1) and (V2) are in fluid communication with a first continuous stirred tank reactor (CSTR1) via conduits (C1) and (C2), respectively. For clarity, the stirring means within the CSTRs in FIG. 1 are not depicted. Vessel (V3) is in fluid communication with a second (CSTR2) via conduit (C3). Each of (CSTR1) through (CSTR10) is connected in series, allowing fluid communication via conduits (C4) through (C12), with (CSTR10) being in fluid communication with quench tank (Q) via conduit (C13). Although the embodiment shown in FIG. 1 comprises ten CSTRs, any number of reactors, although preferably at least three, may be used. In the embodiment shown in FIG. 1, a continuous flow (F1) of the stock preparation of the compound of Formula (2) travels from vessel (V1) through conduit (C1) to (CSTR1) and mixes with a continuous flow (F2) of the stock preparation of the Lewis acid from vessel (V2) to form a combined continuous flow (F1-2) of the two reactants. The combined continuous flow (F1-2) passes through conduit (C4) to (CSTR2) where it mixes with a continuous flow (F3) of the stock preparation of the hydride donor reducing agent that is delivered from vessel (V3) to (CSTR2) through conduit (C3). The resulting continuous flow (F4) passes through the remaining CSTR units via the respective conduits and into the non-continuous flow quench tank (Q) where the continuous flow is collected and the process is quenched. In this embodiment, a supplemental continuous flow (F3′) of a stock preparation of the hydride donor reducing agent from vessel (V3′) is added via conduit (C3′) to (CSTR6) to ensure reaction completion. Alternatively, supplemental continuous flow (F3′) could be added to any CSTR downstream from CSTR2. The flow of the solutions from vessels (V1), (V2), (V3) and (V3′) is facilitated through in-line pumps (P1), (P2), (P3) and (P3′), respectively.

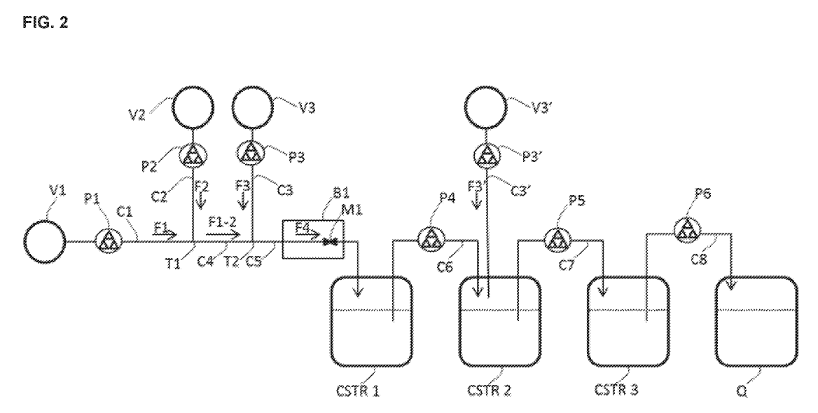

An alternative embodiment shown in FIG. 2, wherein heat generation upon contact of continuous flows (F1), (F2) and (F3) can be reduced. In this alternative embodiment, contact of the flows occurs at the intersections of the conduits carrying the continuous flows rather than in the reactor units.

In FIG. 2, vessel (V1) is connected by conduit (C1) to a first three-way joint (T1), where it intersects with conduit (C2), which is connected to vessel (V2). The downstream conduit (C4) from three-way joint (T1) is connected to a second three-way joint (T2), where it intersects with conduit (C3), which is connected to vessel (V3). Three-way joint (T2) is connected to (CSTR1) via downstream conduit (C5), which is equipped with a magnetic mixer (M1), and which passes through temperature regulation bath (B1). Three continuous stirred tank reactors (CSTR1) through (CSTR3) are connected in series via conduits (C6) and (C7), with (CSTR3) connected to quenching tank (Q) via conduit (C8). For clarity, the stirring means within the CSTRs in FIG. 2 are not depicted. In this arrangement, heat dissipation using external cooling baths and/or mixing units is optimized by conducting the exothermic step (the mixing of continuous flows (F1-2) and (F3)) in a conduit having a high surface area to volume ratio.

In the embodiment shown in FIG. 2, a continuous flow (F1) of the stock preparation of the compound of Formula (2) from vessel (V1) is pushed by pump (P1) through conduit (C1) to the first three-way joint (T1), where it mixes with a continuous flow (F2) of the stock preparation of the Lewis acid pushed from vessel (V2) by pump (P2) via conduit (C2) to form a combined continuous flow (F1-2) comprising the two reactants. Combined continuous flow (F1-2) passes through downstream conduit (C4) from the first three-way joint (T1) to the second three-way joint (T2) where it mixes with a continuous flow (F3) of the stock preparation of the hydride donor reducing agent, which is pushed from vessel (V3) by pump (P3) via conduit (C3). The resulting continuous flow (F4) passes through downstream conduit (C5) where it is mixed with magnetic mixer (M1) and cooled via temperature regulation bath (B1) prior to entering (CSTR1). Continuous flow (F4) is pushed through (CSTR1) and the remaining CSTR units via the respective conduits (C6), (C7) and (C8) by the respective pumps (P4), (P5) and (P6), followed by quenching in non-continuous flow quench tank (Q). In this embodiment, a supplemental continuous flow (F3′) of the stock preparation of the hydride donor reducing agent from vessel (V3′) is added to (CSTR2) by pump (P3′) via conduit (C3′) to ensure reaction completion.

Other alternative configurations of the continuous flow system are also suitable. Preferably, the continuous flow system comprises a combination of conduits and reactors (preferably CSTRs). Usage of a series of one or more reactors in the flow system is preferred. More preferably, three or more CSTRs are used in series as part of the continuous flow system.

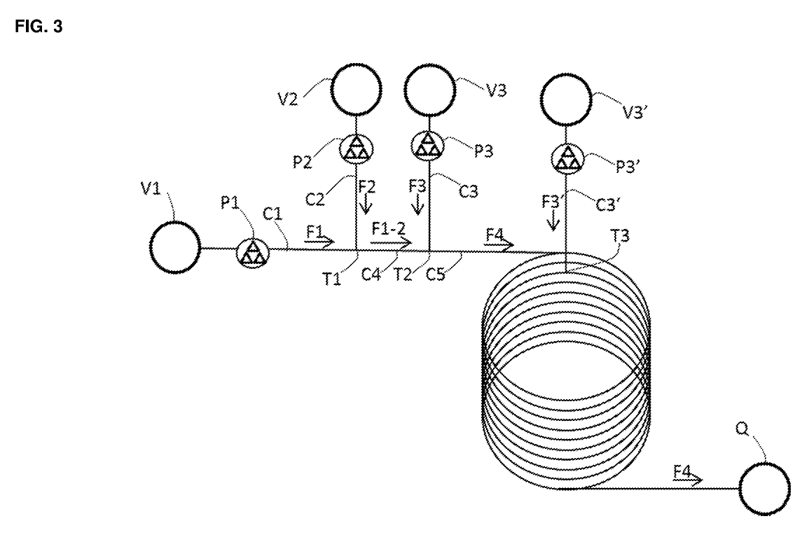

Alternatively, the process of the present invention may be implemented in a continuous flow system comprising a PFR using a number of intersecting conduits without tank reactors as shown in FIG. 3. In the configuration shown, the configuration of components of the system leading to provision of the continuous flow (F4) is analogous to that shown in FIG. 2, however the continuous stirred tank reactors are replaced by conduit (C5) which extends from three-way joint (T2) to the quench tank (Q). A supplemental continuous flow (F3′) of a stock preparation of the hydride donor reducing agent in vessel (V3′) is optionally added by pump (P3′) via conduit (C3′) through three-way joint (T3) in conduit (C5), to ensure reaction completion.

In the processes of the present invention, variables related to the execution of the processes are typically optimized by first determining the time required to achieve reaction completion at a given temperature. This reaction time can then be used to establish the necessary residence time, defined as the time it takes to traverse from contact of continuous flows to quench of the reaction components, in the continuous flow system. The volume of the continuous flow system and the prescribed residence time establish the necessary flow rate of (F4), and the continuous flows (F1), (F2), (F3), and, when used, (F3′). Depending upon the choice of reactants, temperatures and scale of the system, the residence time of the processes of the present invention may be in the range of from about 5 minutes to about 1 hour, and is preferably from about 5 minutes to about 40 minutes.

Preferably, reactants are provided as a stock preparation in a vessel (for example, (V1), (V2), (V3) and (V3′) in FIGS. 1 to 3). However, when reactants are liquid, it is also possible to provide stock preparations by combining the reactant and solvent within a conduit. Any vessel that is compatible with the contents, and which can be equipped with a conduit for export of the contents, is suitable for use in the continuous flow system. It is preferred that stock preparations of the Lewis acid and the hydride donor reducing agent are clear solutions, however a light suspension of reactant is acceptable in so far as it doesn't interfere with the flow of materials, for example, by causing plugging or clogging. The stock preparation of the compound of Formula (2) is typically a stable suspension that does not cause plugging or clogging or otherwise interfere with flow. Some reactants, such as borane, are supplied as a prepared solution in solvent, and are conveniently used as is unless a different concentration is desired.

A separate stock preparation of each reactant is preferably provided for use in the present invention. However, it is also possible to prepare a mixed stock preparation of two compatible reactants, if desired. For example, the compound of Formula (2) and the Lewis acid can be prepared as a single stock preparation, in which case, the continuous flow of (F1) and (F2) is a combined continuous flow (F1-2). Alternatively, pre-combination of two compatible reactants such as the compound of Formula (2) and the Lewis acid is provided by intersecting two distinct continuous flows (F1) and (F2) of each reactant to provide a new continuous flow (F1-2).

In the compound of Formula (2), X is preferably selected from the group consisting of chloride, bromide and iodide. Most preferably, X is chloride.

Solvent (S1) is any suitable solvent capable of forming a stable suspension with the compound of Formula (2) that resists rapid settling, is easily re-dispersed, and flows through the continuous flow system at the desired operational temperature, which is typically from about −5° C. to about 60° C. Preferably, solvent (S1) is selected from the group consisting of ethers and aromatic hydrocarbons. More preferably, solvent (S1) is selected from the group consisting of tetrahydrofuran, diglyme, tetraglyme and toluene. Most preferably, solvent (S1) is tetrahydrofuran.

The concentration of the compound of Formula (2) in solvent (S1) in continuous flow (F1) is a practical concentration which maintains good flowability. Preferably, this concentration is from about 0.6 M to about 1.4 M, more preferably between about 0.90 M and about 1.05 M, even more preferably between about 0.95 M and about 1.05 M, and most preferably about 1.0 M.

The optimal flow rate of continuous flow (F1) is determined taking into consideration the desired stoichiometry of the reaction, the prescribed residence time, and the total volume of the continuous flow system. The compound of Formula (2) is the limiting material in the present invention.

The Lewis acid is any suitable Lewis acid capable of facilitating reductive deoxygenation of a 3-ketoindole in the presence of a hydride reducing agent. Preferably, the Lewis acid is selected from the group consisting of aluminium(III) chloride, iron(III) chloride, magnesium chloride, zinc chloride and boron trifluoride. More preferably, the Lewis acid is selected from the group consisting of aluminium(III) chloride and iron(III) chloride, and is most preferably iron(III) chloride.

Solvent (S2) is any suitable solvent capable of providing complete or substantial dissolution of the Lewis acid at the desired operational temperature, which is typically from about −5° C. to about 60° C. Preferably, solvent (S2) is an ether solvent. More preferably, solvent (S2) is selected from the group consisting of tetrahydrofuran, diglyme and tetraglyme, and is most preferably tetrahydrofuran.

The concentration of the Lewis acid in solvent (S2) in continuous flow (F2) is a practical concentration which maintains dissolution of the Lewis acid. Preferably, the concentration is from about 0.7 M to about 2.0 M, more preferably from about 1.0 M to about 1.25 M, even more preferably from about 1.1 M to about 1.2 M, and most preferably about 1.1 M.

The flow rate of continuous flow (F2) is determined taking into consideration the desired stoichiometry of the reaction, the prescribed residence time, and the total volume of the continuous flow system. Preferably, the molar ratio of the Lewis acid with respect to the compound of Formula (2) is from about 1:1 to about 1.4:1, more preferably from about 1:1 to about 1.3:1, and most preferably is about 1.2:1.

The hydride donor reducing agent is a suitable agent capable of donating two hydride equivalents to facilitate reductive deoxygenation of a 3-ketoindole in the presence of a Lewis acid. Preferably, the hydride donor reducing agent is selected from the group consisting of lithium aluminium hydride, potassium borohydride, sodium borohydride, sodium cyanoborohydride, triethylsilane, borane, diisobutylaluminium hydride and sodium bis(2-methoxyethoxy)aluminium hydride. Most preferably, the hydride donor reducing agent is selected from the group consisting of borane and sodium borohydride.

Solvents (S3) and (S3′) are independently any suitable solvent capable of providing complete or substantial dissolution of the hydride donor reducing agent at the desired operational temperature, which is typically from about −5° C. to about 60° C. Preferably, the solvents (S3) and (S3′) are ethers selected from the group consisting of tetrahydrofuran, diglyme and tetraglyme, and are most preferably tetrahydrofuran or tetraglyme. Preferably, when used, solvent (S3′) is the same as solvent (S3).

The concentration of the hydride donor reducing agent in solvent (S3) or (S3′) in continuous flow (F3) or (F3′), respectively, is a practical concentration which maintains dissolution of the hydride donor reducing agent. Preferably, the concentration is from about 0.75 M to about 2.25 M, more preferably from about 1.0 M to about 2.1 M, and most preferably about 1.9 M.

The flow rate of continuous flow (F3) or (F3′) is determined taking into consideration the desired stoichiometry of the reaction, the prescribed residence time, and the total volume of the continuous flow system. Preferably, the molar ratio of the hydride donor reducing agent in continuous flow (F3) with respect to the compound of Formula (2) in continuous flow (F1) is from about 1:1 to about 1.3:1, more preferably the molar ratio is about 1:1 to about 1.2:1, and most preferably the molar ratio is about 1.1:1. Preferably, the molar ratio of the hydride donor reducing agent in continuous flow (F3′) with respect to the compound of Formula (2) in continuous flow (F1) is from about 0.1:1 to about 0.6:1, and more preferably the molar ratio is from about 0.2:1 to about 0.4:1.

In embodiments, the present invention provides passage of a continuous flow through a conduit. Suitable conduits are any compatible tubing, piping or channel for transmission of organic solutions. In some cases, lengths of conduit that are compatible for use with a peristaltic pump such as Masterflex® peristaltic tubing are used in combination with other types of conduit. Preferably, conduit is constructed of material selected from the group consisting of perfluoroalkoxy alkanes (PFA), polytetrafluoroethylene (PTFE), high density polyethylene (HDPE), Tygon® and Norprene®. Suitable inner diameter and length of conduit is selected to permit unobstructed flow of the contents.

The temperature of the flow of reactants in continuous flows (F1), (F2), (F3), (F3′) and reaction mixture in continuous flow (F4) can be modulated throughout the process as necessary to suit the particular reaction conditions. Due to the physical separation of phases of the reaction that is possible with continuous flow, the temperature is preferably adjusted at different phases to optimize reaction. For example, any exotherm that occurs upon combination of the reactants is preferably extinguished by applying a cooling means to that specific phase of the flow, whereas the temperature of a downstream phase of the flow is adjusted to the optimal reaction temperature for that phase of the process. Due to the excellent temperature control afforded by the high surface area to volume ratio in the continuous flow system, safety concerns are minimized, and reaction efficiency is greatly enhanced by this targeted temperature control. Preferably, any exotherm generated by combination of continuous flows (F1), (F2) and (F3) is controlled by cooling the continuous flow (F4) immediately following mixing to a temperature of from about 10° C. to about 20° C. Otherwise, in the absence of exotherm and/or upon dissipation of the exotherm, the temperature in the remainder of the downstream path of continuous flow (F4) is preferably from about 20° C. to about 60° C., most preferably from about 25° C. to about 40° C.

At the end of the reaction, the continuous flow (F4) is collected into a non-continuous flow quenching tank (Q). The quenching solution is any suitable agent capable of quenching hydride donor reducing agents. Preferably, the quenching solution is a dilute acid solution, preferably wherein the acid is selected from the group consisting of mineral acids and organic acids. Preferably, the quenching solution is aqueous citric acid.

### EXAMPLES

The following examples are illustrative of some of the embodiments of the invention described herein. It will be apparent to the person skilled in the art that various alterations to the described processes in respect of the reactants, reagents and conditions may be made when using the processes of the present invention without departing from the scope or intent thereof. In particular, while the following examples refer to the use of a specific continual flow system design, it will be apparent to the person skilled in the art that different continual flow system designs could alternatively be used.

In the synthetic preparations described in the following Examples, all operations were conducted under a positive nitrogen pressure.

### Analysis Methods Used in the Exemplified Embodiments

The HPLC method described in Table 1 was used to determine the purity (as area %) of the compound of Formula (1-A) in the following Examples.

TABLE 1
HPLC method for the determination of purity
of the compound of Formula (1-A)

| Instrument | Waters 2695 HPLC | | |
|---|---|---|---|
| Column | X-bridge C18, 4.6 × 150 mm, 3.5 μm | | |
| Column Temp. | 35° C. | | |
| Sample temp. | 20-25° C. | | |
| Mobile phase | Solution A: 0.5 mL of 85% H₃PO₄ in 1000 mL of de- | | |
| | ionized water, pH-adjusted to 5.0 ± 0.05 using 5N | | |
| | NaOH solution, filtered and degassed. | | |
| | Solution B: HPLC grade acetonitrile, filtered and | | |
| | degassed. | | |
| | Gradient | | |
| Mode | Time | % Solu- | % Solu- |
| | (min) | tion A | tion B |
| | 0.0 | 75 | 25 |
| | 0.5 | 75 | 25 |
| | 5.0 | 55 | 45 |
| | 12.0 | 56 | 44 |
| | 28.0 | 52 | 48 |
| | 31.0 | 40 | 60 |
| | 36.0 | 40 | 60 |
| | 45.0 | 30 | 70 |
| | 45.0 | 30 | 70 |
| | 48.1 | 75 | 25 |
| | 55.0 | 75 | 25 |

| | |
|---|---|
| Flow rate | 1.0 mL/minute |
| Injection | 10 μL |
| volume | |
| Detector | 240 nm |
| Run time | 55 minutes |
| Sample prep. | Dissolved about 3-5 mg of sample in about 3 mL HPLC |
| | grade methanol. |

### Example 1: Preparation of the Compound of Formula (1-A) with Sodium Borohydride/Iron(III) Chloride Using a Continuous Flow System

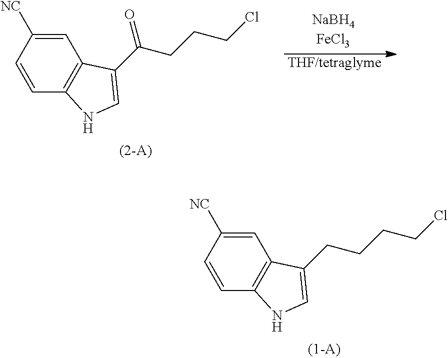 [SMILES: N#Cc1ccc2[nH]cc(C(=O)CCCCl)c2c1 ; "THF/tetraglyme" ; N#Cc1ccc2[nH]cc(CCCCCl)c2c1]

### A. Stock Preparation of the Compound of Formula (2-A)

The compound of Formula (2-A) (20 g, 81.09 mmol, 1.0 eq.) was combined with tetrahydrofuran (68 mL) in a 100 mL round bottomed flask (V1) and the mixture was stirred at room temperature to afford a light suspension (85 mL, 0.95 M).

### B. Stock Preparation of Iron(III) Chloride

Iron(III) chloride (14.47 g, 89.20 mmol, 1.1 eq.) was charged under nitrogen to a 100 mL two-necked round bottomed flask containing tetrahydrofuran (56 mL) at such a rate as to maintain the temperature of the solution during the exothermic addition at less than 30° C. The resulting green solution (78 mL, 1.14 M) was transferred via cannula to a conical flask (V2).

### C. Stock Preparation of Sodium Borohydride

Sodium borohydride (4.60 g, 121.64 mmol, 1.5 eq.) was combined with tetraglyme (60 mL) in a 100 mL round bottomed flask and the mixture was stirred at room temperature to afford a solution (65 mL, 1.87 M). One portion of the solution (47.7 mL, 89.20 mmol, 1.1 eq. NaBH₄) was transferred to a first conical flask (V3). The remainder of the solution (17.3 mL, 32.44 mmol, 0.4 eq. NaBH₄) was transferred to a second conical flask (V3′).

### D. Preparation of the Compound of Formula (1-A)

A continous flow process was conducted in a Coflore® ACR (‘Agitated Cell Reactor’) system by AM Technologies (Runcorn, England) comprising a Digital ACR Agitating Platform (ACR-P200) and a Hastelloy® Reactor Block Assembly (ACR-100-Ha). As depicted in FIG. 1, the Reactor Block had ten 10 mL cells functioning as CSTR units (CSTR1) to (CSTR10). Within the Reactor Block, the cells are joined by channels. (CSTR1) to (CSTR5) were equipped with a 50% volume agitator (i.e., the agitator occupies 50% of the cell volume) and (CSTR6) to (CSTR10) were equipped with a 30% volume agitator (i.e., the agitator occupies 30% of the cell volume), such that the total void volume of the cells within the Reactor Block was 60 mL. Of this void volume, it was estimated that about 60-68%, or 36-40 mL, was occupied by liquid materials, which constituted the ‘working volume’ of the Reactor Block, while the balance was occupied by gaseous by-product.

The stock preparations were pumped by passing a section of the feed conduit through a peristaltic pump. Each conduit (C1), (C2), (C3) and (C3′), carrying continuous flows (F1), (F2), (F3) and (F3′), respectively, and the conduit (C13) leading to the quenching flask (Q), consisted of PFA tubing, other than a short section passing through the corresponding peristaltic pump, which was Masterflex® Chem-Durance Bio Pump L/S 14 peristaltic tubing connected in line with the PFA tubing. The conduits joining the CSTR units were provided as channels within the Reactor Block.

The stock preparations of the compound of Formula (2-A), iron(III) chloride, and sodium borohydride were connected to the Reactor Block as follows: (V1) was connected to (CSTR1) via conduit (C1), (V2) was connected to (CSTR1) via conduit (C2), (V3) was connected to (CSTR2) via conduit (C3), and (V3′) was connected to (CSTR6) via conduit (C3′). Each of conduits (C1), (C2), (C3) and (C3′) was then primed with the respective stock solution. The Reactor Block was heated to 40° C. via circulation of silicon oil from a heating unit.

Stock preparation of the compound of Formula (2-A) from (V1) was pumped as continuous flow (F1) at a flow rate of 0.514 mL/min into (CSTR1) along with the stock preparation of iron(III) chloride from (V2), which was pumped into (CSTR1) as continuous flow (F2) at a flow rate of 0.472 mL/min. When the combined continuous flow (F1-2) began to enter (CSTR2), the primary stock preparation of sodium borohydride in (V3) was pumped into (CSTR2) as continuous flow (F3) at a flow rate of 0.289 mL/min, at which point hydrogen gas evolution began, and the reaction mixture became an orange/green solution, which passed from (CSTR2) as continuous flow (F4) through (CSTR3), (CSTR4) and (CSTR5) into (CSTR6). When continuous flow (F4) started to enter (CSTR6), the stock preparation of sodium borohydride in (V3′) was pumped into (CSTR6) as supplemental continuous flow (F3′) at a flow rate of 0.105 mL/min.

After passing through (CSTR6) through (CSTR10), continuous flow (F4) passed out of (CSTR10) into quench flask (Q) containing an aqueous citric acid solution (0.67 M, 60 mL). At the set flow rates and working volume, the residence time of the process was about 30 minutes. Following consumption of the stock preparations, flasks (V1), (V2) and (V3) were replenished with tetrahydrofuran, which was pumped through the system from each respective flask for 30 minutes at the established flow rates. The combined rinses were collected in quench flask (Q) together with the reaction mixture.

The resulting biphasic solution was concentrated in vacuo to remove tetrahydrofuran, and diluted with n-butanol (60 mL) and water (40 mL) prior to phase separation. The organic phase was washed three times with water (40 mL), diluted with n-butanol (40 mL), and concentrated in vacuo to 40 mL. After concentration, the solution was diluted once more with n-butanol (60 mL) and re-concentrated in vacuo to 40 mL, at which time some solids precipitated. To the resulting suspension was charged concentrated (34-37%) HCl (8.19 g, 81.09 mmol, 1.0 eq.), and the mixture was heated to 65° C. for 3 hours, then slowly cooled to room temperature and maintained at this temperature for 14 hours. After this time, the suspension was filtered, washed with 10 mL cold (−10 to −5° C.) n-butanol and dried in vacuo to provide the compound of Formula (1-A) (6.80 g, 45.6% yield) having HPLC purity of 99.64%.

### Example 2: Preparation of the Compound of Formula (1-A) with Borane/Iron(III) Chloride Using a Continuous Flow System (Flow Configuration 1)

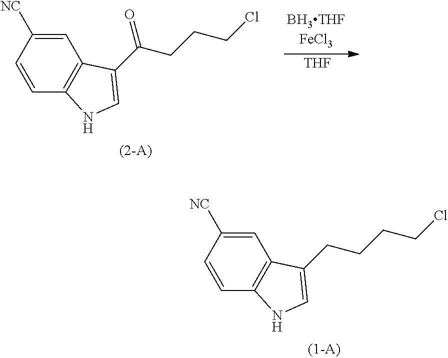 [SMILES: N#Cc1ccc2[nH]cc(C(=O)CCCCl)c2c1 ; N#Cc1ccc2[nH]cc(CCCCCl)c2c1 ; "THF"]

### A. Stock Preparation of the Compound of Formula (2-A)

The compound of Formula (2-A) (20 g, 81.09 mmol, 1.0 eq.) was combined with tetrahydrofuran (73 mL) in a 100 mL round bottomed flask (V1) and the mixture was stirred at room temperature to afford a light suspension (90 mL, 0.90 M).

### B. Stock Preparation of Iron(III) Chloride

Iron(III) chloride (13.15 g, 81.09 mmol, 1.0 eq.) was charged to a 100 mL two-necked round bottomed flask containing tetrahydrofuran (68 mL) at such a rate as to maintain the temperature of the solution during the exothermic addition at less than 30° C. The resulting green solution (70 mL, 1.16 M) was transferred via cannula to a conical flask (V2).

### C. Stock Preparation of Borane

A commercial solution of borane in tetrahydrofuran (1 M) was used. One portion of this solution (81 mL, 81 mmol, 1.0 eq. BH₃) was transferred to a conical flask (V3). A second portion of this solution (16 mL, 16 mmol, 0.2 eq.) was transferred to a separate conical flask (V3′).

### D. Preparation of the Compound of Formula (1-A)

The continous flow process of this example was conducted using the same reactor system as described in Example 1.

The stock preparations of the compound of Formula (2-A), iron(III) chloride, and borane were connected to the Reactor Block as follows: (V1) was connected to (CSTR1) via conduit (C1), (V2) was connected to (CSTR1) via conduit (C2), (V3) was connected to (CSTR2) via conduit (C3), and (V3′) was connected to (CSTR6) via conduit (C3′). Each of conduits (C1), (C2), (C3) and (C3′) was then primed with the respective stock solution. The reactor block was maintained at 20° C. via circulation of silicon oil from a heating unit.

Stock preparation of the compound of Formula (2-A) from (V1) was pumped as continuous flow (F1) at a flow rate of 2.451 mL/min into (CSTR1) along with the stock preparation of iron(III) chloride from (V2), which was pumped into (CSTR1) as continuous flow (F2) at a flow rate of 1.907 mL/min. When the combined continuous flow (F1-2) began to enter (CSTR2), the primary stock preparation of borane in (V3) was pumped into (CSTR2) as continuous flow (F3) at a flow rate of 2.206 mL/min, at which point hydrogen gas evolution began, and the reaction mixture became a yellow/green solution, which passed from (CSTR2) as continuous flow (F4) through (CSTR3), (CSTR4) and (CSTR5) into (CSTR6). When the continuous flow (F4) started to enter (CSTR6), the stock preparation of borane in (V3′) was pumped into (CSTR6) as supplemental continuous flow (F3′) at a flow rate of 0.436 mL/min.

After passing through the remaining CSTRs ((CSTR6) through (CSTR10)), continuous flow (F4) passed out of (CSTR10) into quench flask (Q) containing an aqueous citric acid solution (0.67 M, 60 mL). At the set flow rates and working volume, the residence time of the process was about 5 minutes. Following consumption of the stock preparations, flasks (V1), (V2) and (V3) were replenished with tetrahydrofuran, which was pumped through the system from each respective flask for 5 minutes at the established flow rates. The combined rinses were collected in quench flask (Q) together with the reaction mixture.

The resulting biphasic solution was separated and the organic phase was concentrated in vacuo to 150 mL. The solution was diluted with n-butanol (20 mL) and water (40 mL) prior to phase separation. The organic phase was washed twice with water (40 mL) and then concentrated in vacuo to 40 mL. After concentration, the solution was diluted with n-butanol twice (40 mL, 60 mL) and re-concentrated in vacuo to 40 mL after each dilution, by which time some solids had precipitated. To the resulting suspension was charged concentrated (34-37%) HCl (8.19 g, 81.09 mmol, 1.0 eq.), and the mixture was heated to 65° C. for 3 hours, then slowly cooled to room temperature and maintained at this temperature for 14 hours. After this time, the suspension was filtered, washed with 10 mL cold (−10 to −5° C.) n-butanol and dried in vacuo to provide the compound of Formula (1-A) (12.54 g, 66.5% yield) having HPLC purity of 99.62%.

### Example 3: Preparation of the Compound of Formula (1-A) with Borane/Iron(III) Chloride (Flow Configuration 2)

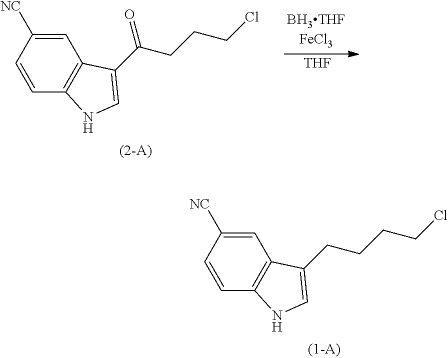 [SMILES: N#Cc1ccc2[nH]cc(C(=O)CCCCl)c2c1 ; N#Cc1ccc2[nH]cc(CCCCCl)c2c1 ; "THF"]

### A. Stock Preparation of the Compound of Formula (2-A)

The compound of Formula (2-A) (30 g, 121.61 mmol, 1.0 eq.) was combined with tetrahydrofuran (105 mL) in a 100 mL round bottomed flask (V1) and the mixture was stirred at room temperature to afford a light suspension (135 mL, 0.90 M).

### B. Stock Preparation of Iron(III) Chloride

Iron(III) chloride (19.73 g, 121.61 mmol, 1.0 eq.) was charged to a 100 mL two-necked round bottomed flask containing tetrahydrofuran (103 mL) at such a rate as to maintain the temperature of the solution during the exothermic addition at less than 30° C. The resulting green solution (105 mL, 1.16 M) was transferred via cannula to a conical flask (V2).

### C. Stock Preparation of Borane

A commercial solution of borane in tetrahydrofuran (1 M) was used. One portion of this solution (121.6 mL, 121.6 mmol, 1.0 eq. BH₃) was transferred to a conical flask (V3). A second portion of this solution (24.3 mL, 24.3 mmol, 0.2 eq.) was transferred to a separate conical flask (V3′).

### D. Preparation of the Compound of Formula (1-A)

A continous flow process was conducted in a system (depicted in FIG. 2) comprised of tubing (conduits), jacketed flasks ((CSTR1), (CSTR2) and (CSTR3), having working volumes of 20 mL, 20 mL and 30 mL, respectively) connected in series via conduits (C6) and (C7), and equipped with magnetic stirring, and quench flask (Q), which was connected to (CSTR3) via conduit (C8). (V1) and (V2), containing the stock solutions of the compound of Formula (2-A) and iron(III) chloride, were connected to a first three-way joint (T1) via conduits (C1) and (C2), respectively. (V3), containing the stock solution of borane), was connected to a second three-way joint (T2) via conduit (C3). The downstream conduit (C4) from three-way joint (T1) was also connected to the second three-way joint (T2). The second three-way joint (T2) was connected to (CSTR1) via downstream conduit (C5), which was equipped with a magnetic mixer (M1), and passed through a water bath maintained at 20° C. (V3′), containing the second portion of the stock borane solution, was connected to (CSTR2) via conduit (C3′). Each of conduits (C1), (C2), (C3) and (C3′) was primed with the respective stock solution.

The stock preparations of the compound of Formula (2-A), iron(III) chloride) and borane were pumped through the respective conduits by passing a section of the conduit through peristaltic pumps (P1), (P2), (P3) and (P3′), respectively. The flow through the three CSTR flasks and into the quenching flask was controlled by passing each connecting conduit through peristaltic pumps (P4), (P5) and (P6), which were activated once the desired working volume in each CSTR was obtained, with the flow rates set to correspond with the incoming flow rates to maintain CSTR volume. Each conduit (C1), (C2), (C3) and (C3′), (C6), (C7) and (C8) consisted of PFA tubing, other than a short section passing through the corresponding peristaltic pump, which was Masterflex® Chem-Durance Bio Pump L/S 14 peristaltic tubing. All other conduits were PFA tubing.

Stock preparation of the compound of Formula (2-A) from (V1) was pumped as continuous flow (F1) at a flow rate of 4.898 mL/min through (C1) towards the first three-way joint (T1). At the same time, stock preparation of iron(III) chloride from (V2) was pumped as continuous flow (F2) at a flow rate of 3.809 mL/min through (C2) towards the first three-way joint (T1). At the first three-way joint (T1), continuous flows (F1) and (F2) joined to form continuous flow (F1-2), which flowed from (T1) through conduit (C4). When the combined continuous flow (F1-2) in (C4) approached the second three-way joint (T2), the primary stock preparation of borane in (V3) was pumped through conduit (C3) as continuous flow (F3) at a flow rate of 4.412 mL/min towards (T2). At the second three-way joint (T2), continuous flows (F1-2) and (F3) join to form continuous flow (F4), which flowed from (T2) through conduit (C5) towards (CSTR1). Continuous flow (F4) passed through conduit (C5), which was submerged in a water bath at 20° C. to control the temperature of the resulting exothermic reaction, and then entered into the first of the three CSTR units. When the continuous flow (F4) of the reaction mixture started to enter (CSTR2), the stock preparation of borane in (V3′) was pumped into (CSTR2) as supplemental continuous flow (F3′) at a flow rate of 0.881 mL/min through conduit (C3′).

The continuous flow (F4) passed from (CSTR2) into (CSTR3), and then out of (CSTR3) into quench flask (Q) containing an aqueous citric acid solution (0.67 M, 60 mL). At the set flow rates and working volume, the residence time of the process was about 5 minutes. Following consumption of the stock preparations in (V1), (V2) and (V3), the flasks were replenished with tetrahydrofuran, which was pumped through the system from each respective flask for 5 minutes at the established flow rates. The combined rinses were collected in quench flask (Q) together with the reaction mixture.

The resulting biphasic solution was separated and the organic phase was concentrated in vacuo to 300 mL. The concentrated organic solution was diluted with n-butanol (60 mL) and water (60 mL), and the phases were separated, with the organic phase being washed twice with water (60 mL) before being concentrated in vacuo to 60 mL, at which time some solid precipitated. After concentration, the suspension was heated to 65° C. and concentrated (34-37%) HCl (12.45 g, 121.61 mmol, 1.0 eq.) was charged, and the mixture was maintained at 65° C. for 3 hours, before being slowly cooled to room temperature and maintained at this temperature for 14 hours. After this time, the suspension was filtered, washed with 15 mL cold (−10 to −5° C.) n-butanol and dried in vacuo to provide the compound of Formula (1-A) (20.16 g, 71% yield) having HPLC purity of 99.60%.

What is claimed is:

1. A continuous flow process for the preparation of the compound of Formula (1): 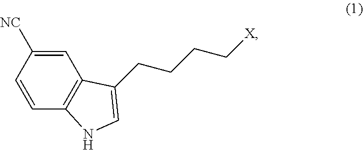
comprising contacting a continuous flow (F1) of the compound of Formula (2): 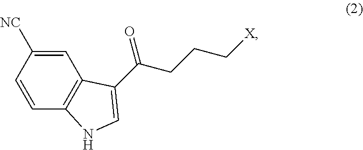
wherein X is a halide,
in a solvent (S1), with a continuous flow (F2) of a Lewis acid in a solvent (S2), and with a continuous flow (F3) of a hydride donor reducing agent in a solvent (S3), to provide continuous flow (F4) containing the compound of Formula (1).

2. The continuous flow process of claim 1, wherein continuous flow (F1) and continuous flow (F2) are combined to form combined continuous flow (F1-2) prior to contact with continuous flow (F3).

3. The continuous flow process of claim 2, wherein continuous flows (F1) and (F2) are combined in a first reactor, and the combined continuous flow (F1-2) that exits the first reactor is contacted with continuous flow (F3) in a second reactor downstream from the first reactor to provide the continuous flow (F4).

4. The continuous flow process of claim 3, wherein continuous flow (F4) passes from the second reactor to a third reactor downstream of the second reactor.

5. The continuous flow process of claim 2, wherein continuous flows (F1) and (F2) are combined at a first intersection to provide combined continuous flow (F1-2) that is then contacted with continuous flow (F3) at a second intersection downstream from the first intersection to provide continuous flow (F4).

6. The continuous flow process of claim 5, wherein continuous flow (F4) passes through one or more reactors connected in series downstream from the second intersection.

7. The continuous flow process of claim 1, wherein continuous flow (F4) is contacted with a supplemental continuous flow (F3′) of the hydride reducing agent in the solvent (S3′).

8. The continuous flow process of claim 1, wherein X is chloride.

9. The continuous flow process of claim 8, wherein the hydride donor reducing agent is selected from the group consisting of borane and sodium borohydride, and the Lewis acid is iron(III) chloride.

10. The continuous flow process of claim 8, wherein each of the solvents (S1), (S2) and (S3), is independently an ether solvent.

11. The continuous flow process of claim 8, wherein the Lewis acid is iron(III) chloride, the hydride donor reducing agent is sodium borohydride, the solvent (S1) and the solvent (S2) are both tetrahydrofuran, and the solvent (S3) is tetraglyme.

12. The continuous flow process of claim 8, wherein the Lewis acid is iron(III) chloride, the hydride donor reducing agent is borane and each of the solvents (S1), (S2) and (S3) is tetrahydrofuran.

13. The continuous flow process of claim 1, wherein continuous flow (F4) is quenched using an aqueous solution prior to isolation of the compound of Formula (1).

14. The continuous flow process of claim 13, wherein the aqueous solution comprises citric acid.

15. A continuous flow system for conducting the continuous flow process of claim 1 comprising:
a first vessel for holding the solution of the compound of Formula (2) in solvent (S1), wherein the first vessel is in fluid communication with one end of a first conduit;
a second vessel for holding the solution of the Lewis acid in solvent (S2), wherein the second vessel is in fluid connection with one end of a second conduit; and
a third vessel for holding the solution of the hydride donor reducing agent in solvent (S3), wherein the third vessel is in fluid communication with one end of a third conduit;
wherein
the first, second and third conduits are merged at their second ends via one or more intersections to provide a fourth conduit that is in fluid communication with a quench tank; and
one or more pumps cause the continuous flow of a first continuous flow of solution from the first vessel, a second continuous flow of solution from the second vessel, and a third continuous flow of solution from the third vessel through the conduits of the continuous flow system to the quench tank.

16. The continuous flow system of claim 15, wherein the first and second conduits are merged at a first intersection to provide a fifth conduit, and the third and fifth conduits are merged at a second intersection to provide the fourth conduit, and wherein each of the intersections comprises either one or more reactors allowing for the mixing of two continuous flows, or three-way joints allowing for the merger of two continuous flows into one continuous flow.

17. The continuous flow system of claim 16, wherein each of the first and second intersections comprises a reactor.

18. The continuous flow system of claim 16, wherein each of the first and second intersections comprises a three-way joint.

19. The continuous flow system of claim 16, wherein the fourth conduit comprises one or more reactors connected in series between the second intersection and the quench tank.

20. The continuous flow system of claim 16, further comprising a supplemental vessel for holding a supplemental portion of the hydride donor reducing agent in solvent (S3′), wherein the supplemental vessel is in fluid communication with a supplemental conduit at one end, and another end of the supplemental conduit merges with the fourth conduit at a third intersection comprising either a reactor or a three-way joint, and a pump causes the continuous flow of solution from the supplemental vessel through the supplemental conduit.
